(19) United States
(12) Patent Application Publication
(10) Pub. No.: US 2026/0108706 A1
(43) Pub. Date: Apr. 23, 2026

(54) INTRALUMINAL BALLOON DILATOR AND ASSOCIATED DEVICES, SYSTEMS, AND METHODS

(71) Applicant: KONINKLIJKE PHILIPS N.V., EINDHOVEN (NL)

(72) Inventors: Nathan C. FRANCIS, COLORADO SPRINGS, CO (US); Kenneth Peter GRACE, WOODLAND PARK, CO (US)

(21) Appl. No.: 19/117,783

(22) PCT Filed: Sep. 22, 2023
(86) PCT No.: PCT/EP2023/076228

§ 371 Date: Apr. 2, 2025

Related U.S. Application Data
(60) Provisional application No. 63/414,073, filed on Oct. 7, 2022.

(51) Int. Cl. A61M 25/06; A61M 25/00; A61M 29/00

(52) U.S. Cl. CPC A61M 25/0662; A61M 25/0023; A61M 29/00; A61M 2210/125

(57) ABSTRACT

An apparatus includes an intraluminal dilator with a flexible elongate member and a balloon. The flexible elongate member can be positioned within a body lumen. The flexible elongate member includes a first diameter. The balloon includes an unexpanded state and an expanded state. In the expanded state, the balloon includes a second diameter larger than the first diameter and a leading edge including a length between a first portion coupled to the flexible elongate member and a second portion with the second diameter. The leading edge extends at an angle between the first diameter and the second diameter. The balloon can engage 2024/074324 an anatomical wall along the leading edge as the intraluminal dilator moves through an opening in the anatomical wall to enlarge the opening such that the second portion of the balloon engages the anatomical wall only after the first portion of the balloon has engaged the anatomical wall.

### TECHNICAL FIELD

The subject matter described herein relates to intraluminal dilators and, in particular, to the introduction of multi-diameter catheters. For example, an intraluminal dilator device may include a balloon dilator tip to introduce multi-diameter catheters through an anatomical wall.

### BACKGROUND

Transcatheter sheaths used in interventional procedures require an atraumatic dilator inserted down the center of the sheath, protruding out the distal end, to safely insert sheath inside blood vessels or across transseptal puncture (TSP) sites within the heart. The distal end of the dilator has a very small tip, usually run over a guidewire, and then gradually gets larger until the proximal end of the dilator matches the inner diameter of the sheath being used. The dilator is uniform in diameter the entire length back to the proximal end. Once the sheath is in place inside the body, the dilator is removed and other tools may be passed down the center of the sheath. However, this requires that the inner diameter of the sheath, the outer diameter of the sheath, and dilator diameter are uniform, creating the smooth transition from the dilator to the sheath.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Disclosed are intraluminal dilator devices, systems, and methods that utilize a balloon dilator to introduce multi-diameter sheath through an anatomical wall. In some instances, the disclosed apparatus gradually enlarges and/or dilates an opening in an anatomical wall utilizing a balloon dilator that is configured to expand to a diameter that is as large as or larger than an inside diameter of a multi-diameter sheath. In some instances, a balloon dilator subsystem controls the expansion of the non-compliant balloon to create a dilating tip that conforms to an internal shape of the multi-diameter catheter. The balloon dilator may be advantageously utilized with multi-diameter sheaths, which may have a larger diameter (e.g., inner diameter and/or outer diameter) at the distal end, compared to smaller diameter more proximally. Existing dilators cannot not be used with multi-diameter sheaths because of the diameter difference between existing dilators and the larger diameter distal end of the for the multi-diameter sheaths. The balloon dilator advantageously expands to makes up for the diameter difference, and provides the balloon dilator a larger diameter at its distal end.

In an exemplary aspect, an apparatus is provided. The apparatus includes an intraluminal dilator comprising: a flexible elongate member configured to be positioned within a body lumen of a patient, wherein the flexible elongate member comprises a first diameter, a proximal portion, and a distal portion; a balloon disposed at the distal portion and comprising an unexpanded state and an expanded state, wherein, in the expanded state, the balloon comprises: a second diameter larger than the first diameter; and a leading edge comprising a length between a first portion coupled to the flexible elongate member and a second portion with the second diameter, wherein the first portion is distal of the second portion such that the leading edge extends at an angle between the first diameter and the second diameter, wherein the balloon is configured to engage an anatomical wall associated with the body lumen along the length of the leading edge as the intraluminal dilator moves through an opening in the anatomical wall to enlarge the opening such that the second portion of the balloon engages the anatomical wall only after the first portion of the balloon has engaged the anatomical wall.

In some aspects, the apparatus includes a fluid source, the flexible elongate member comprises a balloon fluid lumen in fluid communication with the fluid source, and the balloon fluid lumen is configured to receive fluid to transition the balloon between the unexpanded state and the expanded state. In some aspects, the fluid source comprises a liquid source such that the balloon in the expanded state is non-compliant. In some aspects, the distal portion of the flexible elongate member terminates at a distal tip, and the balloon is spaced from and proximal to the distal tip. In some aspects, the distal tip is tapered, and the distal tip is configured to engage the anatomical wall as the intraluminal dilator moves through the opening to enlarge the opening such that the balloon engages the anatomical wall only after the distal tip has engaged the anatomical wall. In some aspects, the angle is between 10 and 20 degrees. In some aspects, the flexible elongate member further comprises a lumen configured to receive at least one of a guidewire or a needle. In some aspects, the intraluminal dilator is configured to be positioned within a sheath comprising a sheath body and sheath tip, wherein the sheath body comprises an inner diameter, the sheath tip comprises an inner diameter, the inner diameter of the sheath tip is larger than the inner diameter of the sheath body, and the balloon is aligned with the sheath tip such that the balloon, in the expanded state, occupies a space within the sheath tip that is defined by the inner diameter of the sheath tip. In some aspects, the apparatus includes the sheath. In some aspects, the balloon comprises a proximal segment and a distal segment, and the proximal segment and the distal segment comprise the second diameter. In some aspects, the balloon comprises a proximal segment and a distal segment, the proximal segment comprises the second diameter, and the distal segment comprises a third diameter larger than the second diameter. In some aspects, the intraluminal dilator is configured to be positioned within a sheath comprising a sheath body and a sheath tip, the sheath body comprises an inner diameter, and the sheath tip comprises an inner diameter and an outer diameter, the inner diameter and the outer diameter of the sheath tip is larger than the inner diameter of the sheath body, the balloon is aligned with the sheath tip such that the proximal segment is positioned inside the sheath tip and the distal segment is positioned outside of the sheath tip, and the third diameter of the distal segment of the balloon is equal to or greater than the outer diameter of the sheath tip.

In an exemplary aspect, a system is provided. The system includes an intraluminal sheath comprising a sheath body and sheath tip, wherein the sheath body comprises an inner diameter, and wherein the sheath tip comprises an inner diameter, wherein the inner diameter of the sheath tip is larger than the inner diameter of the sheath body; and an intraluminal dilator comprising: a flexible elongate member configured to be positioned within a body lumen of a patient, wherein the flexible elongate member comprises a first outer diameter, a proximal portion, and a distal portion; a balloon disposed at the distal portion and comprising an unexpanded state and an expanded state, wherein, in the expanded state, the balloon comprises: a second outer diameter larger than the first diameter; and a leading edge comprising a length between a first portion coupled to the flexible elongate member and a second portion with the second outer diameter, wherein the first portion is distal of the second portion such that the leading edge extends at an angle between the first outer diameter and the second outer diameter, wherein the balloon is configured to engage an anatomical wall associated with the body lumen along the length of the leading edge as the intraluminal dilator moves through an opening in the anatomical wall to enlarge the opening such that the second portion of the balloon engages the anatomical wall only after the first portion of the balloon has engaged the anatomical wall, wherein the balloon is aligned with the sheath tip such that the balloon, in the expanded state, occupies a space within the sheath tip that is defined by the inner diameter of the sheath tip.

In some aspects, the system includes a fluid source, wherein the flexible elongate member comprises a balloon fluid lumen in fluid communication with the fluid source, wherein the balloon fluid lumen is configured to receive fluid to transition the balloon between the unexpanded state and the expanded state.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic, schematic view of a system according to aspects of the present disclosure.

FIG. 2 is a schematic diagram of a processor circuit according to aspects of the present disclosure.

FIGS. 3A and 3B illustrate diagrammatic cross-sectional views of an intraluminal dilator according to aspects of the present disclosure.

FIG. 4A is a diagrammatic side view of a sheath according to aspects of the present disclosure.

FIG. 4B is a diagrammatic cross-sectional side view of the sheath tip disposed at the end of a sheath according to aspects of the present disclosure.

FIG. 5 is a top view of an intraluminal dilator positioned within a sheath according to aspects of the present disclosure.

FIG. 6 illustrates a diagrammatic cross-sectional view of an intraluminal dilator positioned within a sheath according to aspects of the present disclosure.

FIG. 7 illustrates a diagrammatic cross-sectional view of an intraluminal dilator according to aspects of the present disclosure.

FIG. 8 illustrates a diagrammatic cross-sectional view of an intraluminal dilator positioned within a sheath according to aspects of the present disclosure.

FIG. 9 illustrates a diagrammatic cross-sectional view of an intraluminal dilator positioned within a sheath according to aspects of the present disclosure.

FIG. 10 is diagram of a human heart with a needle creating an initial opening in an anatomical wall according to aspects of the present disclosure.

FIG. 11 is diagram of a human heart with a guidewire passing through an initial opening an anatomical wall according to aspects of the present disclosure.

FIG. 12 is a diagrammatic side view of an intraluminal dilator in a sheath being advanced through an anatomical wall to enlarge an initial open according to aspects of the present disclosure.

FIG. 13 illustrates a cross-sectional view of a sheath body of a sheath with a flexible elongate member and a guidewire and/or needle, as seen along the lines of the section A-A of FIG. 12 taken therein, according to aspects of the present disclosure.

FIG. 14 illustrates a cross-sectional view of a sheath body of a sheath with a flexible elongate member and a guidewire and/or needle, as seen along the lines of the section B-B of FIG. 12 taken therein, according to aspects of the present disclosure.

FIG. 15 is diagram of a human heart with balloon dilator and sheath passed through an anatomical wall according to aspects of the present disclosure.

FIG. 16 is diagram of a human heart with a sheath that passed through an anatomical wall according to aspects of the present disclosure.

### DETAILED DESCRIPTION

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

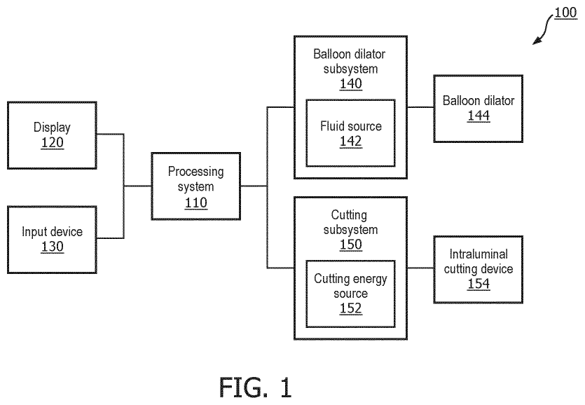

Referring to FIG. 1, shown is a diagrammatic, schematic view of a system according to aspects of the present disclosure. The system 100 may be configured to evaluate (e.g., assess), display, and/or control (e.g., modify) one or more aspects of dilating or enlarging an opening in tissue of a patient's body, e.g., a blood vessel, an anatomical wall, etc., in an atraumatic or non-traumatic way. For instance, the system 100 may be utilized to monitor and/or control crossing transseptal puncture (TSP) sites within a human heart such that the TSP site is traversed without causing traumatic tissue damage, as described in greater detail below. As illustrated, the system 100 may include a processing system 110 in communication with a display device 120 (e.g., an electronic display or monitor), an input device 130 (e.g., a user input device, such as a keyboard, mouse, joystick, microphone, and/or other controller or input device), a balloon dilator subsystem 140 comprising a fluid source 142 and coupled to a balloon dilator 144, and/or a cutting subsystem 150 comprising a cutting energy source 152 and coupled to an intraluminal cutting device 154. In some instances, the intraluminal cutting device 154 may be a cutting sheath, e.g., a laser cutting sheath or a mechanical cutting sheath. In some instances, the cutting energy source 152 may be a laser source configured to provide laser light to the intraluminal cutting device 154 via optical fibers. In some instances, the cutting energy source 152 may be electrical, mechanical, and/or electromechanical energy source to power a mechanical cutting sheath. In general, the device 154 can be any type of intraluminal sheath (e.g., an introducer sheath, a diagnostic sheath, a pure access sheath), and need not to be a cutting sheath. In general, the subsystem 150 can be any type of sheath subsystem. In some instances, the subsystem 150 can be omitted when the device 154 does not need such a subsystem.

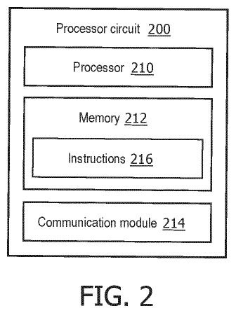

The processing system 110 is generally representative of any device suitable for performing the processing and analysis techniques disclosed herein. In some aspects, the processing system 110 includes a processor circuit, such as the processor circuit 200 of FIG. 2. In some aspects, the processing system 110 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processing system 110 (e.g., computing device) using corresponding instructions stored on or in a non-transitory computer readable medium accessible by the computing device. In some instances, the processing system 110 is a console device. Further, it is understood that in some instances the processing system 110 includes one or a plurality of computing devices, such as computers, with one or a plurality of processor circuits. In this regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

In some instances, the processing system 110 is not in communication with the subsystem 140 and/or the balloon dilator 144. For example, the balloon dilator 144 can be an analog device/catheter, such as a hydraulic device/catheter. The balloon(s) of the balloon dilator 144 can be inflated and deflated (fluid going from fluid source 142 to the balloon dilator 144 for inflation and fluid going from balloon dilator 144 to the fluid 142 for deflation) under manual control of the user using the hydraulics of the balloon dilator subsystem 140 and/or the balloon dilator 144.

FIG. 2 is a schematic diagram of a processor circuit according to aspects of the present disclosure. The processor circuit 200 may be implemented in and/or as part of the processing system 110 of FIG. 1. As shown, the processor circuit 200 may include a processor 210, a memory 212, and a communication module 214. These elements may be in direct or indirect communication with each other, for example via one or more buses.

The processor 210 may include a central processing unit (CPU), a digital signal processor (DSP), an ASIC, a controller, an FPGA, another hardware device, a firmware device, or any combination thereof configured to perform the operations described herein. The processor 210 may also be implemented as a combination of computing devices, e.g., a combination of a DSP and a microprocessor, a plurality of microprocessors, one or more microprocessors in conjunction with a DSP core, or any other such configuration.

The memory 212 may include a cache memory (e.g., a cache memory of the processor 210), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In some instances, the memory 212 includes a non-transitory computer-readable medium. The memory 212 may store instructions 216. The instructions 216 may include instructions that, when executed by the processor 210, cause the processor 210 to perform the operations described herein with reference to the processing system 110 (FIG. 1). Instructions 216 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 214 may include any electronic circuitry and/or logic circuitry to facilitate direct or indirect communication of data between various components of the processor circuit 200 and/or the processing system 110 (FIG. 1). Additionally or alternatively, the communication module 214 may facilitate communication of data between the processor circuit 200, the display device 120, the input device 130, the balloon dilator subsystem 140, the cutting subsystem 150, and/or the like. In this regard, the communication module 214 may be an input/output (I/O) device interface, which may facilitate communicative coupling between the processor circuit 200 and (I/O) devices, such as the input device 130. Moreover, the communication module 214 may facilitate wireless and/or wired communication between various elements of the processor circuit 200 and/or the devices and systems of the system 100 using any suitable communication technology, such as a cable interface such as a USB, micro-USB, Lightning, or FireWire interface, Bluetooth, Wi-Fi, ZigBee, Li-Fi, or cellular data connections such as 2G/GSM, 3G/UMTS, 4G/LTE/WiMax, or 5G.

Turning back now to FIG. 1, the fluid source 142 may be a source for liquid and/or gas. In some instances, the liquid source in the fluid source 142, may be sterile saline, sterile water, oxygen, etc. The fluid source 142 is configured to provide fluid to fill/inflate the balloon of the balloon dilator 144. The fluid source 142 may be configured to receive fluid to empty/deflate the balloon of the balloon dilator 144. In that regard, the balloon dilator subsystem 140 may be configured to provide and/or remove fluid from fluid source 142 to the balloon dilator 144. In some instances, the balloon dilator subsystem 140 may be a pump. For instance, during a first time period, the balloon dilator subsystem 140 may provide sterile saline from the fluid source 142 to inflate a balloon of the balloon dilator 144 such that the balloon engages an anatomical wall associated with the body lumen of the patient as the balloon dilator 144 moves through an opening in the anatomical wall to enlarge the opening. In some aspects, as the opening in the anatomical wall is enlarged to the full diameter of the balloon, the intraluminal cutting device 154 is introduced through the opening into an adjacent body lumen of the patient. In some instances, during a different, second time period, the balloon dilator subsystem 140 may remove the sterile saline from the fluid source 142 to deflate a balloon of the balloon dilator 144. In some instances, once the intraluminal cutting device 154 is introduced through the opening into an adjacent body lumen of the patient and the balloon of the balloon dilator 144 is deflated, the balloon dilator 144 is removed. In some instances, the cutting subsystem 150 is configured to utilize the cutting energy source 152 to operate the intraluminal cutting device 154.

In some aspects, one or more of the balloon dilator subsystem 140 including the fluid source 142, the balloon dilator 144, the cutting subsystem 150 including the cutting energy source 152, and/or the intraluminal cutting device 154 are located spaced from one or more of the processing system 110, the display device 120, and/or the input device 130, such as in different procedure rooms or facilities. For example, the balloon dilator subsystem 140 and/or the cutting subsystem 150 may be part of different systems that are communicatively coupled. In this regard, the processing system 110 may be configured to acquire the data collected from the components spaced therefrom and process the data as described herein.

The balloon dilator subsystem and/or the cutting subsystem 150 may be configured to transmit the collected data to the processing system 110.

The system 100 includes a display device 120 that is communicatively coupled to the processing system 110. In some aspects, the display device 120 is a component of the processing system 110, while in other aspects, the display device 120 is distinct from the processing system 110. In some aspects, the display device 120 is a monitor integrated in a console device or a standalone monitor (e.g., a flat panel or flat screen monitor). The processing system 110 may be configured to generate a visual display (e.g., screen display) based on imaging data from an imaging device. The processing system 110 may provide (e.g., output) the screen display to the display device 120. To that end, the display device 120 may be configured to output (e.g., display) a two-dimensional image and/or a two-dimensional representation of the heart, blood vessels, and/or other anatomy, which may be included in the screen display. In some aspects, the display device 120 is configured to output a three-dimensional graphical representation of the heart, blood vessels, and/or other anatomy. Any suitable display device is within the scope of this disclosure, including self-contained monitors, projection/screen systems, head-up display systems, etc.

The system 100 includes an input device 130 that is communicatively coupled to the processing system 110. The input device 130 may be a peripheral device, such as a touch sensitive pad, a touch-screen, a joy-stick, a keyboard, mouse, trackball, a microphone, an imaging device, and/or the like. In other aspects, the user interface device is part of the display device 120, which may be a touch-screen display, for example. Moreover, a user may provide an input to the processing system 110 via the input device 130. In particular, the input device 130 may enable a user to control, via inputs to the processing system 110, one or more of the components of the system 100, such as the balloon dilator subsystem 140, the balloon dilator 144, the cutting subsystem 150, the intraluminal cutting device 154, or the processing system 110 itself. Additionally or alternatively, the input device 130 may facilitate interaction with a screen display provided at the display device 120. For instance, a user may select, edit, view, or interact with portions of the screen display (e.g., a GUI) provided at the display device 120 via the input device 130.

The system 100 may include various connectors, cables, interfaces, connections, etc., to communicate between the elements of the balloon dilator subsystem 140, the cutting subsystem 150, the intraluminal cutting device 154, the processing system 110, the display device 120, and/or the input device 130. In some aspects, for example, the communication module 214 (FIG. 2), which may be included in the processing system 110, may include such connectors, interfaces, and/or the like. In this regard, the processing system 110 may communicate and/or control one or more components of the processing system 110 via mechanical and/or electromechanical signaling and/or controls. Further, the illustrated communication pathways are exemplary in nature and should not be considered limiting in any way. In this regard, it is understood that any communication pathway between the components of system 100 may be utilized, including physical connections (including electrical, optical, and/or fluid connections), wireless connections, and/or combinations thereof. In this regard, it is understood that the one or more of the components of the system 100 may communicate via a wireless connection in some instances. In some instances, the one or more components of the system 100 and/or other systems (e.g., of a hospital or health services provider) communicate via a communication link over a network (e.g., intranet, internet, telecommunications network, and/or other network).

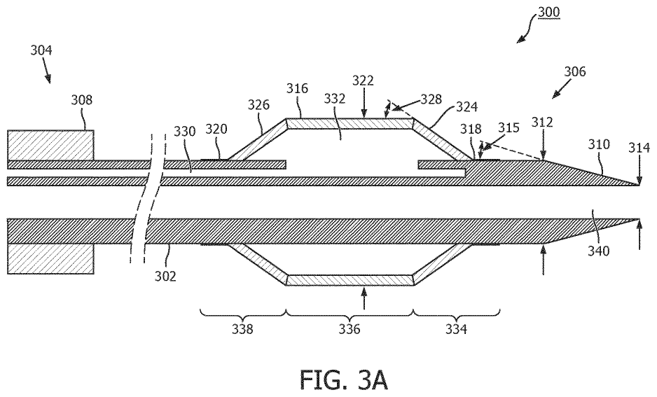

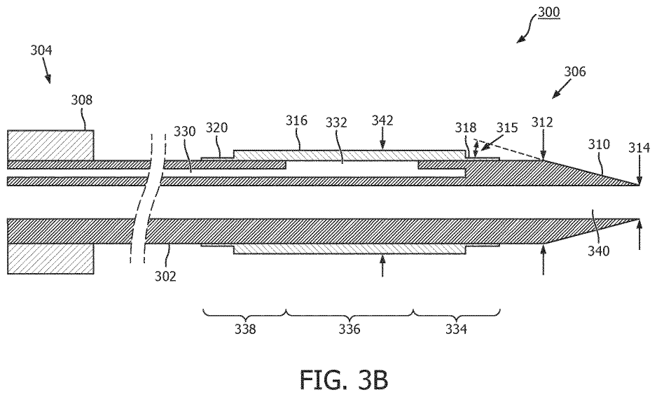

FIGS. 3A and 3B illustrate diagrammatic cross-sectional views of an intraluminal dilator 300 according to aspects of the present disclosure. The intraluminal dilator 300, which may also be referred to as an obturator, may include a flexible elongate member 302 that is configured to be positioned within a body lumen of a patient. The flexible elongate member 302 includes a proximal portion 304 and a distal portion 306. The flexible elongate member 302 includes a handle 308 coupled to and disposed at the proximal portion 304. The flexible elongate member 302 may form at least part of the body of the intraluminal dilator 300. For example, the distal portion 306 and a majority of the length of the flexible elongate member 302 may be positioned within the patient body during use. The proximal portion 304 (e.g., the handle 308) may remain outside of the patient while the distal portion 306 is positioned inside of the patient. The distal portion 306 of the flexible elongate member 302 includes and terminates at a distal tip 310 that is tapered at an angle 315 from a first diameter 312 down to a smaller, second diameter 314. In some instances, the angle 315 of the taper is between 1 degree and 45 degrees. In some instances, the angle 315 of the taper is between 1 degree and 30 degrees. In some instances, the angle 315 of the taper is between 1 degree and 20 degrees. In some instances, the angle 315 of the taper is between 10 degrees and 30 degrees. In some instances, the angle 315 of the taper is between 10 degrees and 20 degrees.

A balloon 316 is disposed at the distal portion 306 of the flexible elongate member 302 spaced from and proximal to the distal tip 310. In some instances, the balloon is formed of a polymer material. Examples of polymer materials are nylon, pebax, polyurethane, and/or polyester. In some instances, the balloon 316 is coupled to the flexible elongate member 302 at a distal end 318 and at a proximal end 320. In some instances, the balloon 316 includes an expanded state (FIG. 3A) and an unexpanded state (FIG. 3B). In some instances, the flexible elongate member 302 includes a balloon fluid lumen 330 in fluid communication with the fluid source 142 of FIG. 1. The balloon fluid lumen 330 is configured to receive fluid to transition the balloon 316 between the unexpanded state and the expanded state via the balloon fluid opening 332. In that regard, the balloon fluid lumen 330 includes a proximal end and a distal end. The proximal end of the balloon fluid lumen 330 is coupled to the fluid source 142 and the distal end of the balloon fluid lumen 330 includes the balloon fluid opening 332 that opens to an inside of the balloon 316 for expanding and deflating the balloon 316. In the expanded state, the balloon 316 includes an expanded diameter 322 that is larger than the first diameter 312 of the flexible elongate member 302. In some instances, the balloon 316 includes a leading edge 324 and a trailing edge 326. The leading edge 324 includes a length between a first portion 334 with the distal end 318 coupled to the flexible elongate member 302 and a second portion 336 with the diameter 322. The trailing edge 326 includes a length between a third portion 338 with the proximal end 320 coupled to the flexible elongate member 302 and the second portion 336 with the diameter 322. In some instances, the first portion at the distal end 318 is distal of the second portion with the expanded diameter 322 such that the leading edge 324 extends at a distal cone angle 328 between the first diameter 312 of the flexible elongate member 302 and the expanded diameter 322 of the balloon 316. In some instances, the angle 328 of the taper is between 1 degree and 45 degrees. In some instances, the angle 328 of the taper is between 1 degree and 30 degrees. In some instances, the angle 328 of the taper is between 1 degree and 20 degrees. In some instances, the angle 328 of the taper is between 10 degrees and 30 degrees. In some instances, the angle 328 of the taper is between 10 degrees and 20 degrees.

In some instances, the distal tip 310 of the flexible elongate member 302 is configured to engage an anatomical wall associated with a body lumen of a patient and create an opening. The intraluminal dilator 300 moves through the opening to enlarge the opening such that the balloon 316 engages the anatomical wall only after the distal tip 310 has engaged the anatomical wall. In that regard, the balloon 316 is configured to continuously engage the anatomical wall associated with the body lumen along the length of the leading edge 324 as the intraluminal dilator 300 moves through the opening in the anatomical wall to enlarge the opening such that the second portion 336 of the balloon 316 engages the anatomical wall only after the first portion 334 of the balloon 316 has engaged the anatomical wall. In some aspects, the flexible elongate member 302 includes a dilator lumen 340 that is configured to receive a guidewire, needle, etc. The dilator lumen 340 can also be referenced as a guidewire lumen. In some instances, the guidewire and/or needle may be passed through the dilator lumen 340 prior to the distal tip 310 engaging the anatomical wall such that the guidewire and/or needle engages the anatomical wall to create an opening smaller than the second diameter 314 of the distal tip 310 and enlarge the opening to the second diameter 314. In that regard, in some instances, the opening is expanded such that the guidewire and/or needle passing through the opening creates an opening that has a diameter at least equal to the second diameter 314, then the distal tip 310 passing through the opening creates an opening that has diameter equal to the first diameter 312, then the balloon 316 passing through the opening creates an opening that has diameter corresponding portion to the diameter 322 of the expanded balloon, thereby creating an opening in an atraumatic or non-traumatic way by gradually enlarging and/or dilating the opening.

FIG. 3B includes features similar to those described in FIG. 3A. In the aspect of FIG. 3B, balloon 316 is shown in a deflated state. In the deflated state, the balloon 136 includes a deflated diameter 342 that is smaller than expanded diameter 322 but is larger than the first diameter 312 of the flexible elongate member 302.

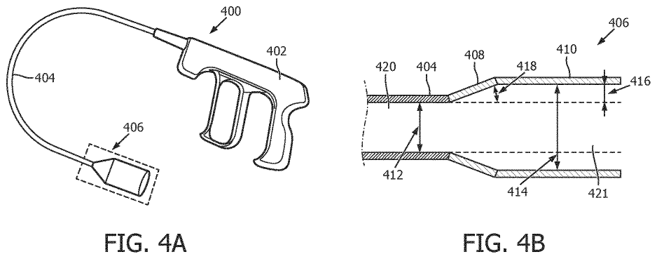

FIG. 4A is a diagrammatic side view of an intraluminal sheath 400 according to aspects of the present disclosure. In some instances, the intraluminal sheath 400 may be a deflectable sheath. In some instances, the intraluminal sheath 400 may be a cutting sheath. In some instances, the cutting sheath may be a laser sheath, a mechanical cutting sheath, etc. In some instances, the cutting sheath may be deflectable. In some instances, the intraluminal sheath 400 may be an introducer sheath, a diagnostic sheath, or a pure access sheath. In some instances, the sheath may be deflectable. In some instances, the intraluminal sheath 400 includes a handle 402, a sheath body 404, and a sheath tip 406. In that regard, a distal end of handle 402 is coupled to a proximal end of sheath body 404 and a distal end of sheath body 404 is coupled to a proximal end of sheath tip 406. In some aspects, the intraluminal dilator 300 of FIGS. 3A and 3B is configured to be positioned within the intraluminal sheath 400. The intraluminal sheath 400 may be referenced as an intraluminal catheter in some instances. As described with respect to FIG. 4B, the intraluminal sheath 400 may be a multi-diameter sheath because of the different diameters of the sheath body 404 and/or the sheath tip 406.

FIG. 4B is a diagrammatic cross-sectional side view of the sheath tip disposed at the end of the intraluminal sheath 400 according to aspects of the present disclosure. In some instances, the sheath tip 406 includes a tapered section 408 that couples a distal section 410 to the sheath body 404. In some aspects, the sheath body 404 includes an inner diameter 412 and the sheath tip 406 includes an inner diameter 414 different than the inner diameter 412. In some instances, the inner diameter 414 of the sheath tip 406 is larger than the inner diameter 412 defining the inner lumen 420 of the sheath body 404. The sheath tip 406 includes inner lumen 421 that is continuous with the inner lumen 420. The dimension 416 is the difference between the inner diameter 414 and the inner diameter 412. The dimension 416 is representative of the diameter difference that would be present if use of an existing dilator was attempted with the sheath 400. Existing dilators would have an outer diameter similar to the diameter 412. Existing dilators cannot be used with the multi-diameter sheath 400 because of the diameter difference 416 prevents a smooth transition between the diameter of existing dilators and the distal sheath 410 of the sheath tip 406.

In some instances, the tapered section 408 extends at an angle 418 from the distal end of the sheath body 404 to the proximal end of the distal section 410 of the sheath tip 406. In some instances, the angle 418 of the taper is between 1 degree and 45 degrees. In some instances, the angle 418 of the taper is between 1 degree and 30 degrees. In some instances, the angle 418 of the taper is between 1 degree and 20 degrees. In some instances, the angle 418 of the taper is between 10 degrees and 30 degrees. In some instances, the angle 418 of the taper is between 10 degrees and 20 degrees. In some instances, the balloon 316 may be aligned with the sheath tip 406 such that the balloon 316, in the expanded state, occupies and/or fills a space within the sheath tip 406 that is defined by the inner diameter 414 of the sheath tip 406.

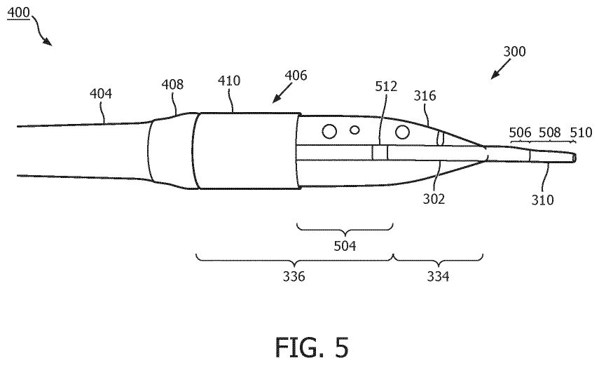

FIG. 5 is a top view of an intraluminal dilator 300 positioned within an intraluminal sheath 400 according to aspects of the present disclosure. In some instances, the sheath tip 406 includes a tapered section 408 that couples a distal section 410 to the sheath body 404. In some instances, a distal segment 504 of the second portion 336 of the balloon 316 may extend distally from the distal section 410. In some aspects, the balloon 316 includes the first portion 334 with a distal end 318 coupled to the flexible elongate member 302 and a proximal end coupled to the second portion 336. In some instances, the distal tip 310 includes one or more tapered sections 506, 508, and 510 so as to gradually enlarge and/or dilate an opening in an anatomical wall. In some instances, the intraluminal dilator 300 may include one or more radiopaque markers, such as radiopaque marker 512, embedded in the flexible elongate member 302 to enable tracking and identify the location of the intraluminal dilator 300 using fluoroscopy.

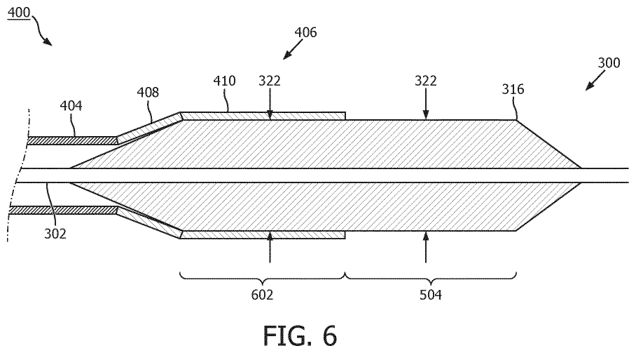

FIG. 6 illustrates a diagrammatic cross-sectional view of an intraluminal dilator 300 positioned within an intraluminal sheath 400 according to aspects of the present disclosure. FIG. 6 includes features similar to those described in FIGS. 3A and 4B. In some instances, the balloon 316 may be aligned with the sheath tip 406 such that the balloon 316, in the expanded state, occupies a space within the sheath tip 406 that is defined by the inner diameter 414 of the sheath tip 406. In some aspects, the balloon 316 includes a proximal segment 602 and a distal segment 504. In some aspects, the proximal segment 602 and the distal segment 504 are expanded such that a diameter of the proximal segment 602 and a diameter of the distal segment 504 are the diameter 322.

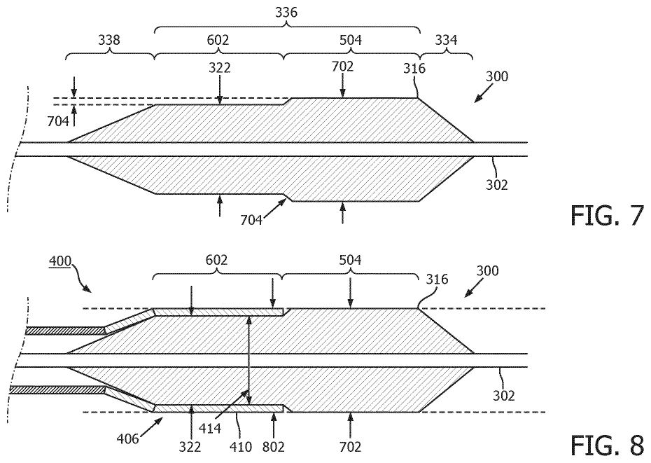

FIG. 7 illustrates a diagrammatic cross-sectional view of an intraluminal dilator 300 according to aspects of the present disclosure. FIG. 7 includes features similar to those described in FIGS. 3A and 6. In some aspects, the balloon 316 includes the first portion 334 with the distal end 318 coupled to the flexible elongate member 302 and a proximal end coupled to the second portion 336, the second portion 336, and the third portion 338 with the proximal end 320 coupled to the flexible elongate member 302 and the second portion 336 with the diameter 322. In some aspects, the balloon 316 includes a proximal segment 602 and a distal segment 504. In contrast to FIG. 6, in some instances, the distal segment 504 may be expanded to a diameter 702 that is larger than diameter 322 by a step dimension 704. In such instances, there is a step dimension 704, which may change in diameter between the distal 322 and the diameter 702.

FIG. 8 illustrates a diagrammatic cross-sectional view of an intraluminal dilator 300 positioned within an intraluminal sheath 400 according to aspects of the present disclosure. FIG. 8 includes features similar to those described in FIGS. 3A, 6, and 7. In some aspects, the balloon 316 may be aligned with the sheath tip 406 such that the balloon 316, in the expanded state, occupies a space within the sheath tip 406 that is defined by the inner diameter 414 of the sheath tip 406. In some aspects, the proximal segment 602 is expanded such that a diameter of the proximal segment 602 is the diameter 322 and a diameter of the distal segment 504 is expanded to the diameter 702 that is equivalent to an outside diameter 802 of the distal section 410 of the sheath tip 406 thus creating a smooth transition between the balloon 316 and the sheath tip 406. In that regard, the inner diameter 414 of the sheath tip 406, which is equivalent to the diameter 322, is smaller than outside diameter 802 of the sheath tip 406, which is equivalent to the diameter 702.

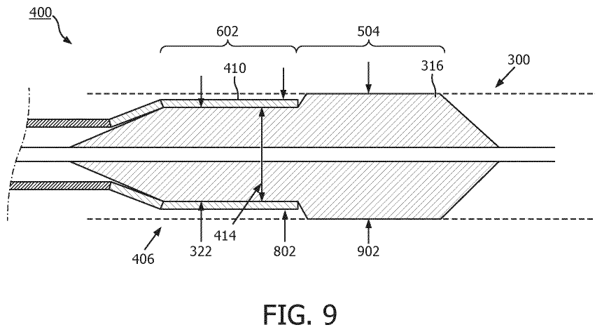

FIG. 9 illustrates a diagrammatic cross-sectional view of an intraluminal dilator 300 positioned within an intraluminal sheath 400 according to aspects of the present disclosure. FIG. 9 includes features similar to those described in FIGS. 3A, 6, and 7. In some aspects, the balloon 316 may be aligned with the sheath tip 406 such that the balloon 316, in the expanded state, occupies a space within the sheath tip 406 that is defined by the inner diameter 414 of the sheath tip 406. In some aspects, the proximal segment 602 is expanded such that a diameter of the proximal segment 602 is the diameter 322. In contrast to FIGS. 6 and 7, a diameter of the distal segment 504 is expanded to a diameter 902 that is greater than the outside diameter 802 of the distal section 410 of the sheath tip 406 thus creating a smooth transition between the balloon 316 and the sheath tip 406. In that regard, the inner diameter 414 of the sheath tip 406, which is equivalent to the diameter 322, is smaller than outside diameter 802 of the sheath tip 406 and smaller than the diameter 902 of the distal segment 504.

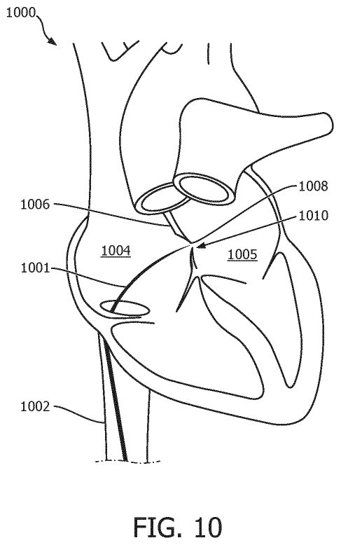

FIG. 10 is diagram of a human heart 1000 with an intraluminal needle 1001 creating an initial opening in an anatomical wall according to aspects of the present disclosure. In some aspects, the intraluminal needle 1001 is inserted through the inferior vena cava 1002 into the right atrium 1004 of the human heart 1000. The right atrium 1004 and the left atrium 1005 are separated from one another by an anatomical wall 1006 that is called the interatrial septum. The intraluminal needle 1001 includes a sharp end 1008, which contacts and is pushed through the anatomical wall 1006 to create an initial opening 1010 in the anatomical wall 1006. The initial opening 1010 can have a relatively small diameter.

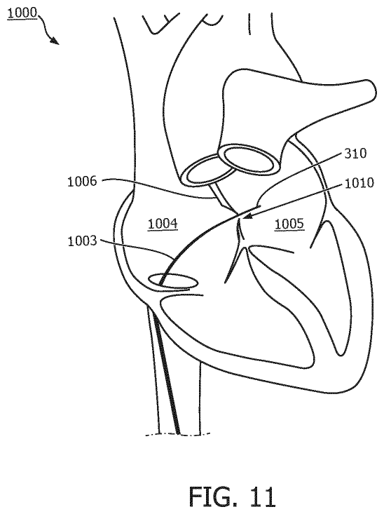

FIG. 11 is diagram of the human heart 1000 with the guidewire 1003 passing through the initial opening 1010 of the anatomical wall 1006 according to aspects of the present disclosure. FIG. 11 includes features similar to those described in FIG. 10. In some aspects, the guidewire 1003 has a relatively small diameter that can fit through and/or be push through the initial opening 1006 in a relatively more atraumatic/non-traumatic manner. In this way, the distal portion of the guidewire 1003 is passed from the right atrium 1004 into the left atrium 1005.

In accordance with the aspects described in FIGS. 1-9, FIG. 12 is a diagrammatic side view of an intraluminal dilator 300 in an intraluminal sheath 400 being advanced through the anatomical wall 1006 to enlarge the initial opening 1010 according to aspects of the present disclosure. The intraluminal dilator 300 and the intraluminal sheath 400 move over the 1003. The distal tip 310 of the intraluminal dilator 300 engages the initial opening 1010 first as the intraluminal dilator 300 is being moved from the right atrium 1004 into the left atrium 1005. Tapered regions of the distal tip 310 expand the diameter and/or cross-sectional area of the opening 1010. As more of the intraluminal dilator 300 passes into the left atrium 1005, the balloon 316 is engages the anatomical wall 1006. For example, the balloon 316 is engages the anatomical wall 1006 along the length of the leading edge as the intraluminal dilator 300 moves through the opening 1010 in the anatomical wall 1006 to enlarge the opening 1010 such that the second portion 336 of the balloon 316 engages the anatomical wall 1006 only after the first portion 334 of the balloon 316 has engaged the anatomical wall 1006. As the balloon 316 moves through the opening 1010, the diameter of the opening 1010 enlarges so that the opening 1010 is at least equal to (and sometimes larger than) the outer diameter of the balloon 316 and/or the sheath tip 406. Before balloon 316 moves through the opening, the diameter of the opening 1010 is smaller than the outer diameter of the balloon 316 and/or the sheath tip 406.

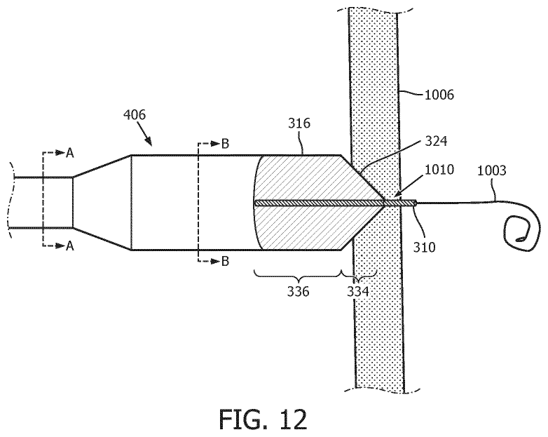

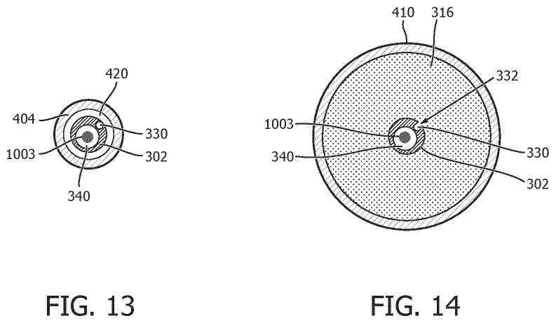

FIG. 13 illustrates a cross-sectional view of the sheath body 404 of the intraluminal sheath 400 with the flexible elongate member 302 and the guidewire and/or needle 1008, as seen along the lines of the section A-A of FIG. 12 taken therein, according to aspects of the present disclosure. In some aspects, the sheath body 404 includes the inner lumen 420 through which the flexible elongate member 302 passes. In some aspects, the flexible elongate member 302 includes the dilator lumen 340 and the balloon fluid lumen 330. In some aspects, the guidewire 1003 passes through the dilator lumen 340.

FIG. 14 illustrates a cross-sectional view of the sheath body 404 of the intraluminal sheath 400 with the flexible elongate member 302 and the guidewire and/or needle 1008, as seen along the lines of the section B-B of FIG. 12 taken therein, according to aspects of the present disclosure. In some aspects, the distal section 410 includes an inner diameter in which the balloon 316, when expanded, occupies a space within the distal section 410. In some aspects, the balloon 316 is coupled to the flexible elongate member 302 that includes the dilator lumen 340 and the balloon fluid lumen 330. In some aspects, the balloon fluid lumen 330 includes the balloon fluid opening 332 that opens to an inside of the balloon 316 for expanding and deflating the balloon 316. In some aspects, the guidewire 1003 passes through the dilator lumen 340.

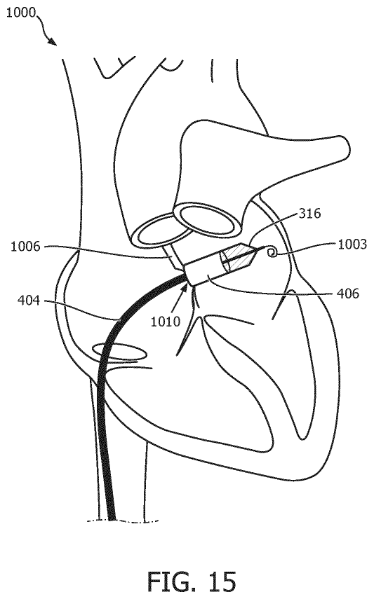

FIG. 15 is diagram of the human heart 1000 with the balloon dilator 300 and the sheath 400 passed through the anatomical wall 1006 according to aspects of the present disclosure. In some aspects, once the balloon 316 advances such that the opening 1010 in the anatomical wall 1006 is enlarged to the diameter of the second portion of the balloon 316, the sheath tip 406, which is coupled to the sheath body 404, is smoothly advanced through the opening 1010.

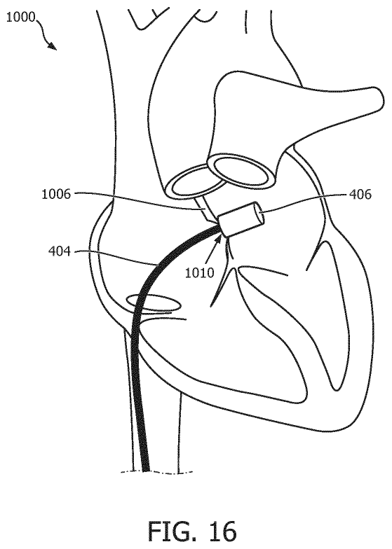

FIG. 16 is diagram of the human heart 1000 with the anatomical wall 1006 according to aspects of the present disclosure. In some aspects, once the sheath tip 406 is advanced through the opening 1010, the balloon 316 is deflated and the balloon 316 and the guidewire 1003 are withdrawn through the sheath tip 406 and the sheath body 404. The sheath tip 406 is then in position for additional steps of therapeutic and/or diagnostic procedures. For example, the sheath tip 406 can be used for cutting tissue. For example, the sheath tip 406 can be in place for delivery of other intraluminal devices.

Accordingly, it may be seen that the disclosed apparatus advantageously enables the introduction of multi-diameter sheaths/catheters through an anatomical wall utilizing a balloon dilator tip. In some instances, the disclosed apparatus gradually enlarges and/or dilates an opening in an anatomical wall utilizing a balloon dilator that is configured to expand to a diameter that is as larger or larger than an inside diameter of a multi-diameter sheath/catheter. Further, it may be seen that the disclosed apparatus advantageously controls the expansion of the non-compliant balloon to create a dilating tip that conforms to a shape of the multi-diameter sheath/catheter.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intraluminal dilator comprising:
a flexible elongate member configured to be positioned within a body lumen of a patient, wherein the flexible elongate member comprises a first diameter, a proximal portion, and a distal portion;
a balloon disposed at the distal portion and comprising an unexpanded state and an expanded state, wherein, in the expanded state, the balloon comprises:
a second diameter larger than the first diameter; and
a leading edge comprising a length between a first portion coupled to the flexible elongate member and a second portion with the second diameter, wherein the first portion is distal of the second portion such that the leading edge extends at an angle between the first diameter and the second diameter,
wherein the balloon is configured to engage an anatomical wall associated with the body lumen along the length of the leading edge as the intraluminal dilator moves through an opening in the anatomical wall to enlarge the opening such that the second portion of the balloon engages the anatomical wall only after the first portion of the balloon has engaged the anatomical wall.

2. The apparatus of claim 1,
further comprising a fluid source,
wherein the flexible elongate member comprises a balloon fluid lumen in fluid communication with the fluid source, wherein the balloon fluid lumen is configured to receive fluid to transition the balloon between the unexpanded state and the expanded state.

3. The apparatus of claim 2, wherein the fluid source comprises a liquid source such that the balloon in the expanded state is non-compliant.

4. The apparatus of claim 1,
wherein the distal portion of the flexible elongate member terminates at a distal tip, and
wherein the balloon is spaced from and proximal to the distal tip.

5. The apparatus of claim 4,
wherein the distal tip is tapered,
wherein the distal tip is configured to engage the anatomical wall as the intraluminal dilator moves through the opening to enlarge the opening such that the balloon engages the anatomical wall only after the distal tip has engaged the anatomical wall.

6. The apparatus of claim 1, wherein the angle is between 10 and 20 degrees.

7. The apparatus of claim 1, wherein the flexible elongate member further comprises a lumen configured to receive at least one of a guidewire or a needle.

8. The apparatus of claim 1,
wherein the intraluminal dilator is configured to be positioned within a sheath comprising a sheath body and sheath tip, wherein the sheath body comprises an inner diameter, and wherein the sheath tip comprises an inner diameter, wherein the inner diameter of the sheath tip is larger than the inner diameter of the sheath body,
wherein the balloon is aligned with the sheath tip such that the balloon, in the expanded state, occupies a space within the sheath tip that is defined by the inner diameter of the sheath tip.

9. The apparatus of claim 8, further comprising:
the sheath.

10. The apparatus of claim 1,
wherein the balloon comprises a proximal segment and a distal segment,
wherein the proximal segment and the distal segment comprise the second diameter.

11. The apparatus of claim 1,
wherein the balloon comprises a proximal segment and a distal segment,
wherein the proximal segment comprises the second diameter, and
wherein the distal segment comprises a third diameter larger than the second diameter.

12. The apparatus of claim 11,
wherein the intraluminal dilator is configured to be positioned within a sheath comprising a sheath body and a sheath tip, wherein the sheath body comprises an inner diameter, and wherein the sheath tip comprises an inner diameter and an outer diameter, wherein the inner diameter and the outer diameter of the sheath tip is larger than the inner diameter of the sheath body,
wherein the balloon is aligned with the sheath tip such that the proximal segment is positioned inside the sheath tip and the distal segment is positioned outside of the sheath tip,
wherein the third diameter of the distal segment of the balloon is equal to or greater than the outer diameter of the sheath tip.

13. A system, comprising:
an intraluminal sheath comprising a sheath body and sheath tip, wherein the sheath body comprises an inner diameter, and wherein the sheath tip comprises an inner diameter, wherein the inner diameter of the sheath tip is larger than the inner diameter of the sheath body; and
an intraluminal dilator comprising:
a flexible elongate member configured to be positioned within a body lumen of a patient, wherein the flexible elongate member comprises a first outer diameter, a proximal portion, and a distal portion;
a balloon disposed at the distal portion and comprising an unexpanded state and an expanded state, wherein, in the expanded state, the balloon comprises:
a second outer diameter larger than the first diameter; and
a leading edge comprising a length between a first portion coupled to the flexible elongate member and a second portion with the second outer diameter, wherein the first portion is distal of the second portion such that the leading edge extends at an angle between the first outer diameter and the second outer diameter,
wherein the balloon is configured to engage an anatomical wall associated with the body lumen along the length of the leading edge as the intraluminal dilator moves through an opening in the anatomical wall to enlarge the opening such that the second portion of the balloon engages the anatomical wall only after the first portion of the balloon has engaged the anatomical wall,
wherein the balloon is aligned with the sheath tip such that the balloon, in the expanded state, occupies a space within the sheath tip that is defined by the inner diameter of the sheath tip.

14. The system of claim 13,
further comprising a fluid source,
wherein the flexible elongate member comprises a balloon fluid lumen in fluid communication with the fluid source, wherein the balloon fluid lumen is configured to receive fluid to transition the balloon between the unexpanded state and the expanded state.
